(19) United States
(12) Patent Application Publication
(10) Pub. No.: US 2003/0144252 A1
(43) Pub. Date: Jul. 31, 2003

(54) Combination product comprising a non-steroidal antiandrogen and an egfr tyrosine kinase inhibitor

(76) Inventor: Barrington John Albert Furr, Macclesfield (GB)

Correspondence Address: MORGAN LEWIS & BOCKIUS LLP, 1111 PENNSYLVANIA AVENUE NW, WASHINGTON, DC, 20004, US

(21) Appl. No.: 10/240,415

(22) PCT Filed: Apr. 3, 2001
(86) PCT No.: PCT/GB01/01537

(30) Foreign Application Priority Data
Apr. 6, 2000 (GB) 0008368.3

(51) Int. Cl. A61K 31/66

(52) U.S. Cl. 514/102

(57) ABSTRACT

The invention concerns a combination therapeutic product comprising a non-steroidal antiandrogen and an epidermal growth factor receptor (EGFR) tyrosine kinase inhibitor (TKI) for use simultaneously, sequentially or separately in the synergistic treatment or prophylaxis of prostate cancer. In particular, the product of the invention is effective in inhibiting the transformation of prostate cancer cells from a hormone-dependent state into a hormone-independent state. It is further expected that the product of the invention will have a beneficial effect in preventing the onset of prostate cancer in men genetically predisposed to the disease.

The present invention relates to a combination therapeutic product comprising an antiandrogen and an epidermal growth factor receptor (EGFR) tyrosine kinase inhibitor (TKI) for use in a new method for the treatment or prophylaxis of prostate cancer. The invention also relates to a pharmaceutical composition comprising such a combination therapeutic product and to the use thereof in the manufacture of a new medicament for use in the treatment or prophylaxis of prostate cancer.

The present invention also relates to the use of such a combination therapeutic product to inhibit the transformation of cancerous cells in the prostate from a hormone-dependent state into a hormone-independent state. In another aspect the invention relates to the use of the combination therapeutic product to inhibit the transformation of prostate cells into cancerous cells i.e. the combination of compounds are prostate cancer chemopreventative agents.

Early and advanced carcinomas of the prostate gland are generally hormone-dependent and, thereby, sensitive, at least for an initial period, to inhibition of androgen-driven growth signalling by way of the androgen receptor. Androgen ablation may be achieved by way of surgical castration or chemical castration, for example using a luteinising hormone releasing hormone (LHRH) agonist such as goserelin or leuprorelin or a LHRH antagonist. The effects of androgens may also be countered using antiandrogen therapy, for example using a non-steroidal antiandrogen such as bicalutamide (or an enantiomer thereof), flutamide and nilutamide. The properties and usefulness of these antiandrogens have been reviewed, for example in the following documents which are incorporated herein by way of reference.

| | |
|---|---|
| bicalutamide | B J A Furr et al., Urology, 1996, 47 (Suppl. 1A), 13-25, |
| | G J C Kolvenbag et al., Urology, 1996, 47 (Suppl. 1A), 70- |
| | 79 and European Patent Application No. 0100172 as the 8th |
| | compound listed in the table in Example 6; |
| flutamide | R O Neri, J. Drug Develop., 1987, 1 (Suppl.), 5-9 and |
| | Urology, 1989, 34 (Suppl. 4), 19-21 and United Kingdom |
| | Patent Application No. 1360001; |
| nilutamide | M G Harris et al., Drugs and Aging, 1993, 3, 9-25 and |
| | United Kingdom Patent Application No. 1518444. |

However, the benefits of androgen ablation or antiandrogen therapy are generally temporary due to the eventual transformation of prostate cancer cells from a hormone-dependent state into a hormone-independent state and/or the clonal selection of androgen-independent prostate cancer cells. It is to be understood that any reference in this patent specification to the inhibition of the transformation of prostate cancer cells from a hormone-dependent state into a hormone-independent state is to be taken as equivalent to a reference to the inhibition of the clonal selection of androgen-independent prostate cancer cells.

In recent years it has been discovered that certain growth factor tyrosine kinase enzymes are important in the transmission of biochemical signals which initiate cell replication. They are large proteins which span the cell membrane and possess an extracellular binding domain for growth factors such as epidermal growth factor (EGF) and an to intracellular portion which functions as a kinase to phosphorylate tyrosine amino acids in proteins and hence to influence cell proliferation.

Various classes of receptor tyrosine kinases are known (Wilks, Advances in Cancer Research, 1993, 60, 43-73) based on families of growth factors which bind to different receptor tyrosine kinases. The classification includes Class I receptor tyrosine kinases comprising the EGF family of receptor tyrosine kinases such as the EGF, TGFα, NEU, erbB, Xmrk, HER and let23 receptors, Class II receptor tyrosine kinases comprising the insulin family of receptor tyrosine kinases such as the insulin and IGFI receptors and insulin-related receptor (IRR) and Class III receptor tyrosine kinases comprising the platelet-derived growth factor (PDGF) family of receptor tyrosine kinases such as the PDGFα, PDGFβ and colony-stimulating factor 1 (CSF1) receptors.

It is known that Class I kinases such as the EGF family of receptor tyrosine kinases are frequently present in common human epithelial cancers such as cancer of the prostate (Visakorpi et al., Histochem. J., 1992, 24, 481). Accordingly it has been recognised that an inhibitor of receptor tyrosine kinases should be of value as a selective inhibitor of the growth of prostate carcinomas.

It is known from European Patent Application No. 0566226 and International Patent Applications WO 96/33980 and WO 97/30034 that certain quinazoline derivatives which possess an anilino substituent at the 4-position possess EGFR tyrosine kinase inhibitory activity and are inhibitors of the proliferation of cancer tissue including prostate cancer. It has been disclosed by J R Woodburn et al. in Proc. Amer. Assoc. Cancer Research, 1997, 38, 633 and Pharmacol. Ther., 1999, 82, 241-250 that the compound N-(3-chloro-4-fluorophenyl)-7-methoxy-6-(3-morpholinopropoxy)quinazolin-4-amine (identified hereinafter by the code number ZD1839) is a potent EGFR TKI.

It is further known from International Patent Application WO 96/30347 that certain structurally-related quinazoline derivatives possessing an anilino substituent at the 4-position also possess EGFR tyrosine kinase inhibitory activity. It has been disclosed in WO 99/55683 that the compoud N-(3-ethynylphenyl)-6,7-bis(2-methoxyethoxy)quinazolin-4-amine, or a pharmaceutically-acceptable salt thereof (linked to the code numbers CP 358774 and OSI-774, identified hereinafter by the code number CP 358774) is an EGFR TKI.

It is further known from International Patent Application WO 97/38983 that certain other structurally-related quinazoline derivatives possessing an anilino substituent at the 4-position also possess EGFR tyrosine kinase inhibitory activity. It has been disclosed in J.Med. Chem., 1999, 42,1803-1815 and WO 00/31048 that the compound 6-acrylamido-N-(3-chloro-4-fluorophenyl)-7-(3-morpholinopropoxy)quinazolin-4-amine (linked to the code numbers PD 183805 and CI 1033, identified hereinafter by the code number CI 1033) is an EGFR TKI.

It is further known from International Patent Application WO 97/02266 that certain other structurally-related heterocyclic derivatives also possess EGFR tyrosine kinase inhibitory activity. For example, the compound 4-[(1R)-1-phenylethylamino]-6-(4-hydroxyphenyl)-7H-pyrrolo[2,3-d]pyrimidine (linked to the code numbers PKI-166, CGP 75166 and CGP 59326, identified hereinafter by the code number PKI-166) is an EGFR TKI.

It is further known from European Patent Application No. 0787722 and International Patent Applications WO 98/50038, WO 99/09016 and WO 99/24037 that certain other structurally-related quinazoline derivatives possessing an anilino substituent at the 4-position also possess EGFR tyrosine kinase inhibitory activity. For example, the compound N-[4-(3-bromoanilino)quinazolin-6-yl]but-2-ynamide (linked to the code numbers CL-387785 and EKB-785, identified hereinafter by the code number CL-387785) is an EGFR TKI.

It is further known from Nature Medicine, 2000, 6, 1024-1028 and U.S. Pat. No. 6,002,008 that certain other structurally-related quinoline derivatives possessing an anilino substituent at the 4-position also possess EGFR tyrosine kinase inhibitory activity. For example, the compound 4-(3-chloro-4-fluoroanilino)-3-cyano-6-(4-dimethylaminobut-2(E)-enamido)-7-ethoxyquinoline (identified hereinafter by the code number EKB-569) is an EGFR TKI.

It is further stated in European Patent Application No. 0566226 and International Patent Application WO 96/33980 that the EGFR TKI compounds disclosed therein may be administered as a sole therapy to provide an anti-proliferative effect or may be administered with one or more other anti-tumour substances, for example cytotoxic or cytostatic anti-tumour substances, for example those selected from, for example, mitotic inhibitors, for example vinblastine, vindesine and vinorelbine; tubulin disassembly inhibitors such as taxol; alkylating agents, for example cis-platin, carboplatin and cyclophosphamide; antimetabolites, for example 5-fluorouracil, tegafur, methotrexate, cytosine arabinoside and hydroxyurea, or, for example, one of the preferred antimetabolites disclosed in European Patent Application No. 239362 such as N-{5-[N-(3,4-dihydro-2-methyl-4-oxoquinazolin-6-ylmethyl)-N-methylamino]-2-thenoyl}-L-glutamic acid; intercalating antibiotics, for example adriamycin, mitomycin and bleomycin; enzymes, for example asparaginase; topoisomerase inhibitors, for example etoposide and camptothecin; biological response modifiers, for example interferon; anti-hormones, for example antioestrogens such as tamoxifen, for example antiandrogens such as 4′-cyano-3-(4-fluorophenylsulphonyl)-2-hydroxy-2-methyl-3′-(trifluoromethyl)propionanilide (bicalutamide) or, for example LHRH antagonists or LHRH agonists such as goserelin, leuprorelin or buserelin and hormone synthesis inhibitors, for example aromatase inhibitors such as those disclosed in European Patent Application No. 0296749, for example 2,2′-[5-(1H-1,2,4-triazol-1-ylmethyl)-1,3-phenylene]-bis(2-methylpropionitrile), and, for example, inhibitors of 5α-reductase such as 17β-(N-tert-butylcarbamoyl)-4-aza-5α-androst-1-en-3-one.

It is disclosed in U.S. Pat. No. 5,985,877 that a combination of a tyrosine kinase inhibitor and chemical castration may be used to treat prostate cancer. The disclosure concerns inhibitors of the tyrosine kinase receptors that bind nerve growth factor, in particular inhibitors of trkA, trkB or trkC. Chemical castration is conventionally achieved by administration of LHRH antagonists or LHRH agonists such as goserelin or leuprorelin. In a related disclosure, D J George et al. in Cancer Research, 1999, 59, 2395-2401 state that a trk tyrosine kinase inhibitor may be combined with surgical castration or with chemical castration obtained using leuprorelin to obtain regression of Dunning R-3327 H rat prostate cancer tissue.

It is disclosed by S Yeh et al. in Proc. Natl. Acad. Sci. USA, 1999, 96, 5458-5463 from study of the LNCaP prostate cancer cell line that cell signalling by way of the HER2/neu protein, a member of the class I or EGFR family of tyrosine kinase enzymes, can activate the androgen receptor in the relative absence of androgen and that this may represent one way by which prostate cancer cells can become transformed from an androgen-dependent to an androgen-independent state. It was noted that the non-steroidal antiandrogen hydroxyflutamide could partially block the HER2/neu protein induced activation of the androgen receptor. It was proposed that inhibitors of the HER2/neu protein could be used to treat prostate cancer.

Similar results have been disclosed by K Griffiths et al. in European Urology, 1997, 32(suppl. 3), 24-40 involving study of the LNCaP prostate cancer cell line. It was noted that the non-steroidal antiandrogen bicalutamide (Casodex, registered trade mark) could partially block EGF induced cell growth. It was proposed that specific tyrosine kinase inhibitors could be used to treat prostate cancer by restraining prostate cancer progression whilst a warning was given of the capacity of cancer cells to circumvent the blockade of a specific signalling pathway by activation of another pathway.

We have now found that unexpectedly the combination use of particular non-steroidal antiandrogens and particular EGFR tyrosine kinase inhibitors in the treatment of prostate cancer can have a synergistic effect in terms of one or more of the extent of the response, the response rate, the time to disease progression and the survival rate. In particular, we have found that the combination use of particular non-steroidal antiandrogens and particular EGFR tyrosine kinase inhibitors is especially effective in inhibiting the transformation of prostate cancer cells from a hormone-dependent state into a hormone-independent state and consequently the combination has a pronounced effect on the time to disease progression and the survival rate. It is further expected that the combination use of particular non-steroidal antiandrogens and particular EGFR tyrosine kinase inhibitors will have a beneficial effect in preventing the onset of prostate cancer in men genetically predisposed to the disease.

It is believed that the beneficial effects of the combination use of particular non-steroidal antiandrogens and particular EGFR tyrosine kinase inhibitors in the treatment of prostate cancer may be due to the functional ‘cross-talk’ between the androgen and EGFR tyrosine kinase signalling pathways which leads to inhibition of the progression of prostate cancer cells from an androgen-dependent to an androgen-independent state. As discussed by L J Denis and K Griffiths in Seminars in Surgical Oncology, 2000, 18, 52-74, it is believed that the binding of the dihydrotestosterone:androgen receptor complex to DNA modifies the shape of adjacent DNA strands to facilitate binding of the relevant transcription factors activated by growth factor signalling to drive cell growth. It will be appreciated that androgen ablation by way of surgical or chemical castration can not influence the ‘cross talk’ phenomenon other than by reduction of testosterone levels and an ensuing reduction in the quantity of dihydrotestosterone:androgen receptor complex. In contrast, given the effects of antiandrogens on growth factor stimulated growth of prostate cancer cell lines as noted above from the studies of Yeh et al. and Griffiths et al., the antiandrogen:androgen receptor to complex can directly or indirectly participate in ‘cross-talk’ between the androgen and growth factor signalling pathways and partially inhibit growth factor driven growth.

In addition, it will be appreciated that androgen ablation by way of surgical or chemical castration can not prevent androgen arising from the adrenal glands from influencing prostate cancer growth. In contrast, antiandrogen therapy antagonises androgen irrespective of its origin in the testes or adrenal glands.

It should therefore be appreciated that there is no equivalency of the concept or effect of the use of a combination comprising an antiandrogen and an EGFR TKI compared to a combination comprising surgical or chemical castration and an EGFR TKI.

According to a first aspect of the present invention there is provided a combination therapeutic product comprising a non-steroidal antiandrogen and an EGFR TKI for use simultaneously, sequentially or separately in the synergistic treatment or prophylaxis of prostate cancer.

The present invention is also capable of being used simultaneously, sequentially or separately in the synergistic treatment or prophylaxis of non-malignant disease of the prostate gland such as benign prostatic hypertrophy (BPH).

In a suitable therapeutic product of the invention, the non-steroidal antiandrogen is selected from, for example, bicalutamide (or an enantiomer thereof), flutamide and nilutamide. Preferably the non-steroidal antiandrogen component of the combination therapeutic product is bicalutamide.

In a suitable therapeutic product of the invention, the EGFR TKI is selected from, for example, ZD1839, CP 358774, CI 1033, PKI-166, CL-387785 and EKB-569. Preferably the EGFR TKI component of the combination therapeutic product is ZD1839 or CP 358774. More preferably the EGFR TKI component of the combination therapeutic product is ZD1839.

It should be appreciated that there is no requirement that the antiandrogen and EGFR TKI components of the therapeutic product of the invention must be dosed simultaneously. Sequential or separate use of these components may also provide the desired beneficial effect and such use is to be understood to fall within the definition of a product of the invention. Factors such as the rate of absorption, metabolism and the rate of excretion of each agent will affect their presence at the tumour site. Such factors are routinely considered by, and are well within the ordinary skill of, the clinician when he contemplates the treatment of a medical condition which requires the conjoint administration of two agents in order to obtain a beneficial effect.

It should also be appreciated that according to the present invention a combination therapeutic product is defined as affording a synergistic effect if the effect is therapeutically superior, as measured by, for example, the extent of the response, the response rate, the time to disease progression or the survival period, to that achievable on dosing one or other of the components of the combination product at its conventional dose. For example, the effect of the combination product is synergistic if the effect is therapeutically superior to the effect achievable with a non-steroidal antiandrogen alone or an EGFR TKI alone. Further, the effect of the combination product is synergistic if a beneficial effect is obtained in a group of patients that does not respond (or responds poorly) to a non-steroidal antiandrogen alone or an EGFR TKI alone. In addition, the effect of the combination product is defined as affording a synergistic effect if one of the components is dosed at its conventional dose and the other component is dosed at a reduced dose and the therapeutic effect, as measured by, for example, the extent of the response, the response rate, the time to disease progression or the survival period, is equivalent to that achievable on dosing conventional amounts of the components of the combination product. In particular, synergy is deemed to be present if the conventional dose of the EGFR TKI component of the combination product may be reduced without detriment to one or more of the extent of the response, the response rate, the time to disease progression and survival data, in particular without detriment to the duration of the response, but with fewer and/or less troublesome side-effects than those that occur when conventional doses of each component are used.

According to a preferred version of this aspect of the present invention there is provided a combination therapeutic product comprising the non-steroidal antiandrogen bicalutamide and the EGFR TKI ZD1839 for use simultaneously, sequentially or separately in the synergistic treatment or prophylaxis of prostate cancer.

A therapeutic product of the invention may be administered in the form of a pharmaceutical composition. According to this aspect of the invention there is provided a pharmaceutical composition for use in the synergistic treatment or prophylaxis of prostate cancer which comprises a non-steroidal antiandrogen and an EGFR TKI in conjunction or admixture with pharmaceutically-acceptable diluents or carriers.

It will be appreciated that the pharmaceutical composition according to the present invention includes a composition comprising a non-steroidal antiandrogen, an EGFR TKI and a pharmaceutically-acceptable diluent or carrier. Such a composition conveniently provides the therapeutic product of the invention for simultaneous use in the synergistic treatment or prophylaxis of prostate cancer.

A pharmaceutical composition according to the present invention also includes separate compositions comprising a first composition comprising a non-steroidal antiandrogen and a pharmaceutically-acceptable diluent or carrier, and a second composition comprising an EGFR TKI and a pharmaceutically-acceptable diluent or carrier. Such a composition conveniently provides the therapeutic product of the invention for sequential or separate use in the synergistic treatment or prophylaxis of prostate cancer but the separate compositions may also be administered simultaneously. Conveniently such a pharmaceutical composition of the invention comprises a kit comprising a first container with a suitable composition containing the non-steroidal anti-androgen and a second container with a suitable composition containing the EGFR TKI.

The compositions of the invention may be in a form suitable for oral use (for example as tablets, capsules, aqueous or oily suspensions, emulsions or dispersible powders or granules), for topical use (for example as creams, ointments, gels, or aqueous or oily solutions or suspensions; for example for use within a transdermal patch), for parenteral administration (for example as a sterile aqueous or oily solution or suspension for intravenous, subcutaneous, intramuscular or intravascular dosing) or as a suppository for rectal dosing. Preferably the compositions of the invention are in a form suitable for oral use, for example as tablets or capsules.

The compositions of the invention may be obtained by conventional procedures using conventional pharmaceutically-acceptable diluents or carriers that are well known in the art.

Suitable pharmaceutically-acceptable diluents or carriers for a tablet formulation include, for example, inert diluents such as lactose, sodium carbonate, calcium phosphate or calcium carbonate, granulating and disintegrating agents such as corn starch or alginic acid; binding agents such as gelatin or starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin or olive oil.

According to a preferred version of this aspect of the present invention there is provided a pharmaceutical composition as defined hereinbefore wherein the non-steroidal antiandrogen is bicalutamide and the EGFR TKI is ZD1839.

The amount of each active ingredient of the present combination therapeutic product in such pharmaceutical compositions will necessarily vary depending upon the host treated and the particular route of administration. For the non-steroidal antiandrogen component, a tablet or capsule formulation intended for oral administration will generally contain, for example, from about 20 mg to 1 g of active ingredient When the non-steroidal antiandrogen component is bicalutamide, a conventional tablet formulation may be used for daily oral administration containing between 50 and 300 mg of active ingredient, conveniently 50 mg, 80 mg, 150 mg or 300 mg of active ingredient, preferably containing 150 mg of active ingredient. For the EGFR TKI component, a tablet or capsule formulation intended for oral administration will also generally contain, for example, from about 20 mg to 1 g of active ingredient. When the EGFR TKI is ZD1839, a conventional tablet formulation may be used for oral administration containing 50 mg, 100 mg, 250 mg or 500 mg of active ingredient. Conveniently the daily oral dose of ZD1839 is above 150 mg, for example, in the range 150 to 750 mg, preferably in the range 200 to 500 mg. For a single dosage form, the active ingredients may be compounded with an appropriate and convenient amount of excipients which may vary from about 5 to about 98 percent by weight of the total composition. Dosage unit forms will generally contain about 20 mg to about 500 mg of each active ingredient. Alternatively each active ingredient may be combined separately with one or more excipients to produce a two-part dosage form. In the latter event the pharmaceutical composition of the invention comprises a kit comprising a first container with a suitable composition containing the non-steroidal anti-androgen and a second container with a suitable composition containing the EGFR TKI. Such a kit may, for example, allow the physician wishing to treat his patient's prostate cancer to select the appropriate amounts of each active ingredient and the sequence and timing of the administration thereof. Those skilled in the art of treating prostate cancer patients can readily select the appropriate conventional amounts of each active ingredient and a suitable dosing schedule.

In a further aspect of the present invention we have found that unexpectedly the conventional dose of the EGFR TKI component of the combination therapeutic product may be reduced without detriment to one or more of the extent of the response, the response rate, the time to disease progression and survival data, in particular without detriment to the duration of the response but with fewer and/or less troublesome side-effects and this is one aspect of the synergistic effect of the present invention. In particular, we have found that with the combination use of the non-steroidal antiandrogen bicalutamide with the EGFR TKI ZD1839 the preferred daily oral dose of ZD1839 of 200 to 500 mg may be reduced to about 150 mg or less, preferably to between 30 and 100 mg, without detriment to the duration of response but with fewer side-effects. The physician wishing to treat his patient's prostate cancer knows how to select the appropriate amount of EGFR TKI such as ZD1839 and the sequence and timing of the administration thereof. For example, in patients with advanced prostate cancer, the physician would use a conventional dose of non-steroidal antiandrogen such as bicalutamide, preferably a 150 mg daily oral dose thereof, and, whilst titrating down the dose of the EGFR TKI, would monitor the individual patient's prostate specific antigen (PSA) level, a drop in PSA being a well-established marker of a beneficial response to treatment of the advanced prostate cancer patient. This beneficial effect may be due to continued inhibition of the transformation of prostate cancer cells from a hormone-dependent state into a hormone-independent state. Consequently this combination retains a pronounced effect on the time to disease progression and survival data while side effects arising from treatment with an EGFR TKI are reduced.

According to this aspect of the invention there is provided a pharmaceutical composition comprising the non-steroidal antiandrogen bicalutamide in a dosage amount of between 50 and 300 mg, preferably 150 mg, and the EGFR TKI ZD1839 in a dosage amount of about 150 mg or less, preferably between 30 and 100 mg, and a pharmaceutically-acceptable diluent or carrier.

Further according to this aspect of the invention there is provided a pharmaceutical composition comprising a first composition comprising the non-steroidal antiandrogen bicalutamide in a dosage amount of between 50 and 300 mg, preferably 150 mg, and a pharmaceutically-acceptable diluent or carrier, and a second composition comprising the EGFR TKI ZD1839 in a dosage amount of about 150 mg or less, preferably between 30 mg and 100 mg, and a pharmaceutically-acceptable diluent or carrier.

According to these two aspects of the invention the non-steroidal antiandrogen bicalutamide is administered in a daily oral dosage amount of between 50 and 300 mg, preferably 150 mg, and the EGFR TKI ZD1839 in a daily oral dosage amount of about 150 mg or less, preferably between 30 and 100 mg.

According to a further aspect of the invention there is provided the use of a combination therapeutic product as defined hereinbefore for the manufacture of a medicament for administration simultaneously, sequentially or separately to a warm-blooded animal such as a human male for the treatment or prophylaxis of prostate cancer.

According to a further aspect of the invention there is provided a method for the treatment or prophylaxis of prostate cancer which comprises the administration simultaneously, sequentially or separately to a warm-blooded animal such as a human male of an effective amount of a combination therapeutic product as defined hereinbefore.

Further the combination therapeutic product of this aspect of the invention may contain an additional step or component which, together with the antiandrogen component already present, provides for total or maximal androgen blockade. According to this aspect of the invention there is provided a combination therapeutic product comprising a non-steroidal antiandrogen, a chemical castration agent and an EGFR TKI for use simultaneously, sequentially or separately in the synergistic treatment or prophylaxis of prostate cancer.

In a suitable therapeutic product of the invention, the chemical castration agent is LHRH, a LHRH agonist such as goserelin or leuprorelin or a LHRH antagonist which agent is administered at its conventional dose using its conventional dosing schedule.

According to a preferred version of this aspect of the present invention there is provided a combination therapeutic product comprising the non-steroidal antiandrogen bicalutamide, a chemical castration agent selected from goserelin and leuprorelin and the EGFR TKI ZD1839 for use simultaneously, sequentially or separately in the synergistic treatment or prophylaxis of prostate cancer.

According to this aspect of the invention there is provided a pharmaceutical composition for use in the synergistic treatment or prophylaxis of prostate cancer which comprises a non-steroidal antiandrogen, a chemical castration agent and an EGFR TKI in conjunction or admixture with pharmaceutically-acceptable diluents or carriers.

According to a further aspect of the invention there is provided the use of a combination therapeutic product as defined immediately hereinbefore for the manufacture of a medicament for administration simultaneously, sequentially or separately to a warm-blooded animal such as a human male for the treatment or prophylaxis of prostate cancer.

According to a further aspect of the invention there is provided a method for the treatment or prophylaxis of prostate cancer which comprises the administration simultaneously, sequentially or separately to a warm-blooded animal such as a human male of an effective amount of a combination therapeutic product as defined immediately hereinbefore.

According to a further aspect of the invention there is provided the combination of surgical castration together with the use of a combination therapeutic product comprising a non-steroidal antiandrogen and an EGFR TKI for the manufacture of a medicament for administration simultaneously, sequentially or separately to a warm-blooded animal such as a human male for the treatment or prophylaxis of prostate cancer.

According to a further aspect of the invention there is provided a method for the treatment or prophylaxis of prostate cancer which comprises the combination of surgical castration together with the administration simultaneously, sequentially or separately to a warm-blooded animal such as a human male of an effective amount of a combination therapeutic product comprising a non-steroidal antiandrogen and an EGFR TKI.

In a second part of the present invention we have found that unexpectedly the combination therapeutic product comprising particular non-steroidal antiandrogens and particular EGFR tyrosine kinase inhibitors has effects not only on the growth of transformed prostate cancer cells but also on the constitutive growth of normal and abnormal, but non-malignant, epithelial or stromal cells in the prostate. It is well established that in epithelial or stromal prostatic tissue, androgen such as testosterone and particularly dihydrotestosterone stimulates normal growth. Constitutive growth of prostatic cells comprises the non-androgen dependent baseline turnover of cells. The combination of particular non-steroidal antiandrogens and particular EGFR tyrosine kinase inhibitors may therefore be used to reduce, preferably to inhibit, the transformation of prostatic cells, in particular prostatic stromal cells, to a malignant state.

It is known that there is a series of changes in the appearance of normal prostatic epithelium as malignancy develops. In normal prostatic epithelium, cells have nuclei of standard size and standard chromatin levels whereas in the early stages of invasive carcinoma of the prostate the cells have markedly enlarged nuclei or nucleoli and also markedly increased chromatin levels. There is an intermediate stage in this disease process, known as prostatic intraepithelial neoplasia (PIN) identified as that stage where, in general, the size of the nuclei has begun to be enlarged and the chromatin levels have begun to be increased. The combination of particular non-steroidal antiandrogens and particular EGFR tyrosine kinase inhibitors of the present invention can inhibit the transformation of normal prostatic cells, in particular prostatic epithelial cells, from a normal to a PIN state. Said combination can also inhibit the transformation of PIN cells to a malignant state.

According to this aspect of the present invention there is provided a combination therapeutic product comprising an antiandrogen and an EGFR TKI for use simultaneously, sequentially or separately in reducing, preferably inhibiting, the transformation of prostatic cells to a malignant state in a human in need of such treatment.

The invention, according to this second part, is particularly beneficial in preventing the onset of prostate cancer in men genetically predisposed to the disease. Conventional methods are available to classify patients according to their risk of contracting prostate cancer, for example by assessment of family history and measurements over time of particular blood proteins such as PSA and assessment of the extent of the presence of PIN.

According to a preferred version of this aspect of the present invention there is provided a combination therapeutic product comprising the non-steroidal antiandrogen bicalutamide and the EGFR TKI ZD1839 for use simultaneously, sequentially or separately in reducing, preferably inhibiting, the transformation of prostatic cells to a malignant state in a human in need of such treatment.

According to this aspect of the invention there is also provided a pharmaceutical composition for use in reducing, preferably inhibiting, the transformation of prostatic cells to a malignant state in a human in need of such treatment which comprises a non-steroidal antiandrogen and an EGFR TKI in conjunction or admixture with pharmaceutically-acceptable diluents or carriers.

According to this aspect of the invention there is also provided a pharmaceutical composition for use in reducing, preferably inhibiting, the transformation of prostatic cells to a malignant state in a human in need of such treatment which comprises the non-steroidal antiandrogen bicalutamide in a dosage amount of between 50 and 300 mg, preferably 150 mg, and the EGFR TKI ZD1839 in a dosage amount of about 150 mg or less, preferably between 30 and 100 mg, and a pharmaceutically-acceptable diluent or carrier.

Further according to this aspect of the invention there is provided a pharmaceutical composition for use in reducing, preferably inhibiting, the transformation of prostatic cells to a malignant state in a human in need of such treatment which comprises a first composition comprising a non-steroidal antiandrogen and a pharmaceutically-acceptable diluent or carrier, and a second composition comprising an EGFR TKI and a pharmaceutically-acceptable diluent or carrier. Preferably the non-steroidal antiandrogen is bicalutamide and the EGFR TKI is ZD1839.

Further according to this aspect of the invention there is provided a pharmaceutical composition for use in reducing, preferably inhibiting, the transformation of prostatic cells to a malignant state in a human in need of such treatment which comprises a first composition comprising the non-steroidal antiandrogen bicalutamide in a dosage amount of between 50 and 300 mg, preferably 150 mg, and a pharmaceutically-acceptable diluent or carrier, and a second composition comprising the EGFR TKI ZD1839 in a dosage amount of about 150 mg or less, preferably between 30 mg and 100 mg, and a pharmaceutically-acceptable diluent or carrier.

According to these three aspects of the invention the non-steroidal antiandrogen bicalutamide is administered in a daily oral dosage amount of between 50 and 300 mg, preferably 150 mg, and the EGFR TKI ZD1839 in a daily oral dosage amount of between 200 and 500 mg, preferably about 150 mg or less, more preferably between 30 and 100 mg.

Further according to this aspect of the invention there is provided the use of a combination therapeutic product as defined hereinbefore for the manufacture of a medicament for administration simultaneously, sequentially or separately to a warm-blooded animal such as a human male for use in reducing, preferably inhibiting, the transformation of prostatic cells to a malignant state in a human in need of such treatment.

Further according to a further aspect of the invention there is provided a method for use in reducing, preferably inhibiting, the transformation of prostatic cells to a malignant state in a human in need of such treatment which comprises the administration simultaneously, sequentially or separately to a warm-blooded animal such as a human male of an effective amount of a combination therapeutic product as defined hereinbefore.

The inhibition of cellular transformation defined hereinbefore involves a combination treatment. It may be beneficial to employ sequential therapy with, for example, a first treatment period of about 1 to 6 months during which a conventional dose of the EGFR TKI such as ZD1839 is administered followed by a second treatment period of about 1 to 6 months during which a conventional dose of the antiandrogen such as bicalutamide is administered. Thereby a period is allowed whereby some constitutive growth of prostate tissue is permitted in order to minimise the extent of tissue atrophy.

Alternatively the combination therapy may include the continuous administration of the antiandrogen component such as bicalutamide and the intermittent administration of the EGFR TKI such as ZD1839. The intermittent therapy of the EGFR TKI may involve, for example, a two-monthly cycle of treatment comprising a first portion involving the dosing of the EGFR TKI for a period of about one month followed by a second portion involving an EGFR TKI drug-free period of about one month. Thereafter further two-monthly cycles of such treatment may be given.

### EXPERIMENTAL METHODS

Both in vitro experimental methods and in vivo experimental methods in animals and/or appropriate clinical trials in human males can be used to assess the activity of the combination therapeutic product of the present invention.

In Vitro Methods

Androgen-dependent or androgen-independent human prostate cancer cell lines can be exposed in vitro to various concentrations of either the antiandrogen or EGF TKI component of the combination product of the present invention or to various concentrations of a combination of both components. Thereby the extent and duration of the effect of the combination can be determined. For example, human prostate DU145 cells, TSU-PR1 cells, CWR22 cells, PC-3 cells or LNCaP cells can be used. Growth inhibition can be assessed using, for example, a standard soft agar colony-forming assay or, for example, a standard MTT assay. Cellular apoptosis can be assessed using, for example, a standard ELISA assay, for example the Cell Death Detection ELISA Plus Kit available from Boehringer, Mannheim, Germany. Thereby it can be shown that, for example, an increased inhibition of cell growth is obtained with a combination of an antiandrogen and an EGFR TKI than the maximum obtainable effect of either component of the combination when used alone at concentrations that are not grossly cytotoxic and, for example, the dose response curve for either component can be shifted to show greater potency when the combination is used.

In Vivo Methods

Tumours derived from prostate cancer tissue or cell lines can be grown in animals such as rats or mice, particularly athymic nude mice or rats. After inoculation or implantation and growth of the tumour cells or tissue, the test animals can be treated with the combination product of the invention and the size of the tumour before, during and after each treatment schedule can be assessed to provide an indication of the therapeutic effect of the treatment.

For example, a xenograft model can be used involving the implantation and growth of Dunning R-3327 H prostate cancer tissue in adult male inbred Copenhagen rats according to the general procedures disclosed by J T Isaacs et al., Cancer Research, 1981, 41, 5070-5075 and Cancer Research, 1989, 49, 6290-6294 and by D J George et al., Cancer Research, 1999, 59, 2395-2401. Test compounds can be suspended in, for example, Tween 80 (registered trade mark) by ball-milling, for example for about 16 hours, and dosed orally by gavage. It can be shown that the oral administration of a combination product of the antiandrogen bicalutamide and the EGFR TKI ZD1839 causes substantial reductions in tissue proliferation, for example as measured by conventional Ki67 immunostaining of excised xenograft tissue, and a substantial and sustained reduction in the tumour growth rate.

For example, using a xenograft model involving the implantation and growth of human CWR22 androgen-dependent prostate cancer in male nude mice according to the general procedures disclosed by T G Pretlow et al., Cancer Research, 1994, 54, 6049-6052 and Cancer Research, 1996, 56, 3042-3046, it can be shown that the oral administration of a combination product of the antiandrogen bicalutamide and the EGFR TKI ZD1839 causes substantial reductions in tissue proliferation, for example as measured by conventional Ki67 immunostaining of excised xenograft tissue, and a substantial and sustained reduction in the tumour growth rate.

For example, using a xenograft model involving the implantation and growth of human PC-3 or TSU-PR1 prostate cancer in male nude mice, it can be shown that the oral administration of a combination product of the antiandrogen bicalutamide and the EGFR TKI ZD1839 causes a substantial and sustained reduction in the tumour growth rate.

Human Clinical Trial

Patients presenting with prostate cancer will be assessed for disease stage and PSA level will be used as an appropriate tumour marker. Patients with appropriate entry criteria will be allocated to the clinical programme. One group of patients will be dosed orally with the antiandrogen bicalutamide and a second group of patients will be dosed orally with a combination of the antiandrogen bicalutamide and the EGFR TKI ZD1839. Blood samples will be taken periodically and analysed for the level of PSA. Localised prostate tumour growth will be assessed using one or more of digital rectal examination (DRE), computer assisted tomography (CAT) scanning and prostate tissue biopsy sampling. Clinical responses will be defined using conventional criteria. For example, a complete response will indicate that the tumour mass has regressed totally, a partial response will be defined as a 50% or greater reduction in the original tumour volume and stable disease will be defined as a reduction in tumour volume of less than 50% or no increase in tumour volume.

A corresponding trial in patients presenting with BPH can also be conducted.

1. A combination therapeutic product comprising a non-steroidal antiandrogen and an epidermal growth factor receptor (EGFR) tyrosine kinase inhibitor (TKI) for use simultaneously, sequentially or separately in the synergistic treatment or prophylaxis of prostate cancer.

2. A combination therapeutic product according to claim 1 comprising the non-steroidal antiandrogen bicalutamide and the EGFR TKI ZD1839 for use simultaneously, sequentially or separately in the synergistic treatment or prophylaxis of prostate cancer.

3. A pharmaceutical composition for use in the synergistic treatment or prophylaxis of prostate cancer which comprises a non-steroidal antiandrogen and an EGFR TKI in conjunction or admixture with pharmaceutically-acceptable diluents or carriers.

4. A pharmaceutical composition according to claim 3 for simultaneous use in the synergistic treatment or prophylaxis of prostate cancer comprising a non-steroidal antiandrogen, an EGFR TKI and a pharmaceutically-acceptable diluent or carrier.

5. A pharmaceutical composition according to claim 3 for simultaneous, sequential or separate use in the synergistic treatment or prophylaxis of prostate cancer comprising a kit comprising a first composition comprising a non-steroidal antiandrogen and a pharmaceutically-acceptable diluent or carrier, and a second composition comprising an EGFR TKI and a pharmaceutically-acceptable diluent or carrier.

6. A pharmaceutical composition according to any one of claims 3 to 5 wherein the non-steroidal antiandrogen is bicalutamide and the EGFR TKI is ZD1839.

7. A pharmaceutical composition according to any one of claims 3 to 5 wherein the non-steroidal antiandrogen is bicalutamide at a daily oral dose between 50 and 300 mg.

8. A pharmaceutical composition according to any one of claims 3 to 5 wherein the EGFR TKI is ZD1839 at a daily oral dose between 200 and 500 mg.

9. A pharmaceutical composition according to any one of claims 3 to 5 wherein the EGFR TKI is ZD1839 at a daily oral dose of about 150 mg or less.

10. A pharmaceutical composition according to claim 3 comprising the non-steroidal antiandrogen bicalutamide in a dosage amount of between 50 and 300 mg and the EGFR TKI ZD1839 in a dosage amount of about 150 mg or less, and a pharmaceutically-acceptable diluent or carrier.

11. A pharmaceutical composition according to claim 3 comprising a first composition comprising the non-steroidal antiandrogen bicalutamide in a dosage amount of between 50 and 300 mg and a pharmaceutically-acceptable diluent or carrier, and a second composition comprising the EGFR TKI ZD1839 in a dosage amount of about 150 mg or less, and a pharmaceutically-acceptable diluent or carrier.

12. The use of a combination therapeutic product according to claim 1 or claim 2 for the manufacture of a medicament for administration simultaneously, sequentially or separately to a warm-blooded animal such as a human male for the treatment or prophylaxis of prostate cancer.

13. A method for the treatment or prophylaxis of prostate cancer which comprises the administration simultaneously, sequentially or separately to a warm-blooded animal such as a human male of an effective amount of a combination therapeutic product according to claim 1 or claim 2 or an effective amount of a pharmaceutical composition according to any one of claims 3 to 11.

14. A combination therapeutic product comprising a non-steroidal antiandrogen, a chemical castration agent and an EGFR TKI for use simultaneously, sequentially or separately in the synergistic treatment or prophylaxis of prostate cancer.

15. A combination therapeutic product according to claim 14 comprising the non-steroidal antiandrogen bicalutamide, a chemical castration agent selected from goserelin and leuprorelin and the EGFR TKI ZD1839 for use simultaneously, sequentially or separately in the synergistic treatment or prophylaxis of prostate cancer.

16. A combination therapeutic product comprising an antiandrogen and an EGFR TKI for use simultaneously, sequentially or separately in reducing the transformation of prostatic cells to a malignant state in a human in need of such treatment.

17. A combination therapeutic product according to claim 16 comprising the antiandrogen bicalutamide and the EGFR TKI ZD1839 for use simultaneously, sequentially or separately in reducing the transformation of prostatic cells to a malignant state in a human in need of such treatment.

18. A pharmaceutical composition for use in reducing the transformation of prostatic cells to a malignant state in a human in need of such treatment which comprises a non-steroidal antiandrogen and an EGFR TKI in conjunction or admixture with pharmaceutically-acceptable diluents or carriers.

19. A pharmaceutical composition according to claim 18 for use in reducing the transformation of prostatic cells to a malignant state in a human in need of such treatment comprising a kit comprising a first composition comprising a non-steroidal antiandrogen and a pharmaceutically-acceptable diluent or carrier, and a second composition comprising an EGFR TKI and a pharmaceutically-acceptable diluent or carrier.

20. A pharmaceutical composition according to claim 18 or claim 19 wherein the non-steroidal antiandrogen is bicalutamide and the EGFR TKI is ZD1839.

21. A pharmaceutical composition according to claim 18 or claim 19 wherein the non-steroidal antiandrogen is bicalutamide at a daily oral dose between 50 and 300 mg.

22. A pharmaceutical composition according to claim 18 or 19 wherein the EGFR TKI is ZD1839 at a daily oral dose between 200 and 500 mg.

23. A pharmaceutical composition according to claim 18 or claim 19 wherein the EGFR TKI is ZD1839 at a daily oral dose of about 150 mg or less.
